(19) United States
(12) Patent Application Publication
(10) Pub. No.: US 2026/0192014 A1
(43) Pub. Date: Jul. 9, 2026

(54) Composite Filament for Surgical Suture

(71) Applicant: Cannuflow, Inc., Scotts Valley, CA (US)

(72) Inventor: Theodore R. Kucklick, Scotts Valley, CA (US)

(73) Assignee: Cannuflow, Inc., Scotts Valley, CA (US)

(21) Appl. No.: 19/438,809

(22) Filed: Jan. 2, 2026

Related U.S. Application Data
(60) Provisional application No. 63/742,153, filed on Jan. 6, 2025.

(51) Int. Cl. A61L 17/12; C08L 89/00

(52) U.S. Cl. CPC A61L 17/12; C08L 89/00; C08L 2203/02

(57) ABSTRACT

A composite filament that is made of collagen and polycaprolactone. The collagen can be either a crosslinked or an uncrosslinked collagen. The composite is formed by combining either the crosslinked or uncrosslinked collagen at a melt temperature that is lower than a denaturing temperature of either the crosslinked or uncrosslinked collagen.

### SUMMARY

This application claims priority to U.S. Provisional Application 63/742,153 filed Jan. 6, 2025.

### FIELD OF THE INVENTIONS

The inventions described below relate to the field of development and production of a composite filament core for use in surgical applications.

### BACKGROUND OF THE INVENTIONS

Surgical sutures, scaffolds, and implantable constructs are used to provide temporary mechanical support and to facilitate tissue repair. Many such devices are formed from synthetic polymers selected for strength and processability. While these materials may provide adequate mechanical performance, they typically do not provide biological cues that promote tissue ingrowth or regeneration.

Collagen is a bioabsorbable material known for its biocompatibility and ability to support cellular attachment. Type I collagen, in particular, is associated with biological responses relevant to tissue healing. However, collagen is thermally sensitive, and uncrosslinked collagen denatures at relatively low temperatures. Exposure to elevated processing temperatures can alter the native structure of collagen and reduce its biological functionality.

Thermoplastic polymers, including polycaprolactone (PCL), are commonly processed using melt-based techniques. Conventional melt processing temperatures for such polymers are generally incompatible with uncrosslinked collagen, thereby limiting the ability to form collagen-polymer composite filaments without degrading the collagen. As a result, some composite materials rely on solvent-based processing or multi-step fabrication methods, which can increase manufacturing complexity and introduce additional constraints.

There remains a need for bioabsorbable composite materials that combine collagen with thermoplastic polymers in a form suitable for filament-based processing, while maintaining processing temperatures below the denaturation temperature of collagen. There is further a need for such materials to be manufacturable without the use of solvents and to be suitable for use as load-bearing components in sutures, scaffolds, and related medical constructs.

### SUMMARY

The devices and methods described below provide for a composite filament core made of collagen and polycaprolactone (PCL) that can be processed at a temperature below the temperature collagen denatures for use in a surgical suture, scaffold or construct. The composite filament can be overbraided over the composite filament. The composite filament does not require solvents to process the polymer in the core.

The composite filament is mechanically compounded and blended together.

The composite filament is a collagen-polycaprolactone composite that can be processed at a temperature below 45° C., which is the temperature uncrosslinked collagen begins to denature. Alternatively, the composite can be processed below 100° C. which is the temperature that crosslinked collagen to denatures. The composite filament can made by either lowering the melt temperature of the polymer or alternatively by raising the thermal resistance of the collagen, or both. The collagen-polycaprolactone filament is advantageous over other filaments because it has type 1 collagen stimulating properties.

### BRIEF DESCRIPTION OF THE DRAWINGS

FIG. 1a illustrates a composite filament for a surgical suture and FIG. 1b illustrates a cross section of the suture.

FIG. 2 illustrates the method of compounding the composite filament core via a melt processing method.

FIG. 3 illustrates the method for extruding the collagen-polycaprolactone filament for additive manufacturing.

FIG. 4 illustrates the suture overbraid method.

FIG. 5 illustrates a method of 3D printing a composite filament for a surgical construct.

FIG. 6 illustrates the collagen particle size to the PCL core size in comparison.

### DETAILED DESCRIPTION OF THE INVENTIONS

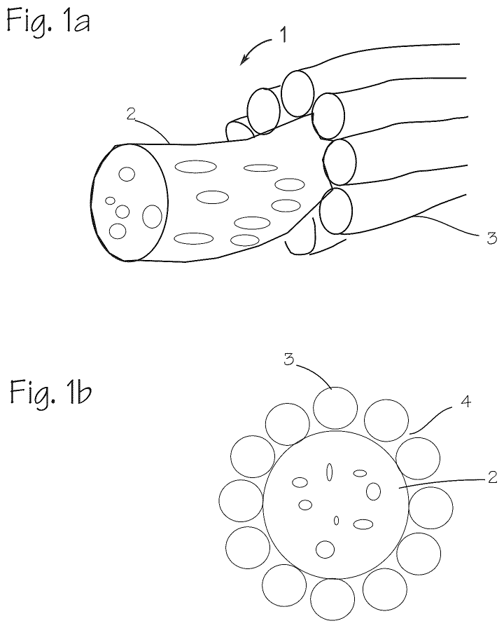

FIG. 1a illustrates a composite filament for a surgical suture 1 and FIG. 1b illustrates a cross section of the surgical suture or construct. A composite filament core 2 is surrounded by braided polyester and/or polyethylene fibers 3 to form the suture. The composite filament is a collagen and polycaprolactone composite that is extruded into a filament that can then be overbraided with polyester and polyethylene fibers to form the suture. The composite filament forms a core of the suture that serves as a scaffold for tissue ingrowth and in addition the braided fibers provide load sharing of the suture. The composite filament is a collagen-polycaprolactone composite that can be processed at a temperature below 45° C., which is the temperature uncrosslinked collagen begins to denature, or under 100° C. if crosslinked collagen is used. The polycaprolactone refers to the entire polycaprolactone family, either unmodified, or polycaprolactone, Poly(ϵ-Caprolactone)-Based Copolymers, poliglecaprone or any in the polycaprolactone family that have been modified to have a melt temperature below 45° C. either by the reduction of molecular weight of addition of plasticizers or other means. The braided fibers include openings 4 between 5 and 300 microns to allow cells to pass through the fibers into the composite filament core. The composite can be used as a scaffold core for a suture. The composite can also be made into a filament for 3D printing. This allows the production of a wide variety of scaffolds, surgical fasteners, tacks, anchors, meshes, and repair devices made with this collagen-polycaprolactone material. Alternatively, the composite can be paired with fiber reinforcement for load-sharing, or in a flat repair mesh. The suture may be round or a flat tape design. The collagen can be bovine or porcine type I or a synthetic recombinant collagen such as Evonik Vecollan®. The composite may also have other bioactive additives, like antibiotics, anti-inflammatories, bone morphogenic proteins (BMP) hyaluronic acid, hydroxyapatite (HA).

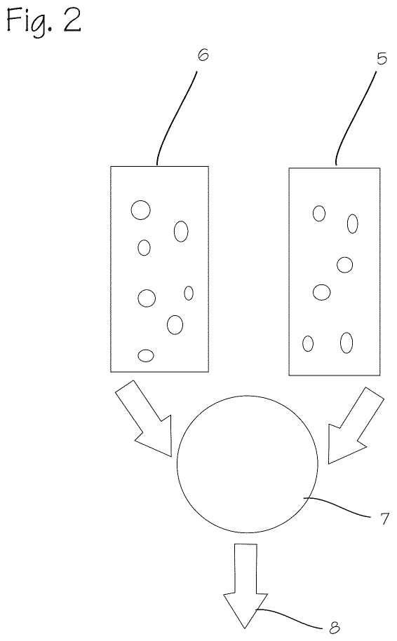

FIG. 2 illustrates the method of compounding the composite filament core via a melt processing method. Micronized freeze dried collagen powder 5 is blended with low melt polycaprolactone pellets that may be modified for low melt temperature 6. The polycaprolactone acts as a binder to the collagen powder and results in a composition that can be extruded into the filament core 2. The polycaprolactone pellets and micronized freeze dried collagen are processed through a compounding mill 7 to produce bulk collagen-polycaprolactone composite 8. A low-melt polycaprolactone pellet has a melting point below 45° C. It is mixed together with a micronized freeze dried collagen powder and processed through a compounding mill to produce a composite that can be extruded into a filament. The low-melt polycaprolactone pellets can be any pellets that have reduced crystallinity to result in a lower melting point, specifically below 45° C. To reduce the melt temperature, the polycaprolactone may add plasticizers such as Dioctyl Phthalate (DOP), Polyethylene glycol (PEG) or Triethyl citrate. Alternatively, the melt temp of polycaprolactone can also be lowered with pressurized CO₂. CO₂-induced melting temperature depression of PCL, PHB and PHBV can be achieved by using a variable-volume high-pressure view-cell equipped with the optical instrumentation for measurement of transmitted light intensities and real time recording as a function of temperature and/or pressure. The melting temperature depression curve of polycaprolactone has a temperature maximum at 0.5 MPa and a temperature minimum at 8 MPa. Its melting temperature can be reduced to 45° C. in CO₂ at pressures at and above 15 MPa. Freeze drying is often referred to as lyophilization. The freeze-drying process can tune the porosity of the collagen product. Collagen can be processed by freeze-drying and ground to make a powdered filler. The particle size is from 5 to 300 micrometers. The collagen can be Type 1 collagen, atelocollagen, recombinant collagen, or vegan synthetic collagen (Evonik Vecollan®).

The collagen may be crosslinked in order to improve the heat resistance. Crosslinked collagen exhibits a higher temperature resistance compared to native collagen because the crosslinks formed between collagen molecules create a more stable network, preventing the fibers from easily separating and denaturing at elevated temperatures; essentially, the added crosslinks “lock” the collagen structure together, increasing its thermal stability. The collagen can be cross linked with crosslinking agents to create chemical bonds between individual collagen molecules. This increased stability means the crosslinked collagen can withstand higher temperatures before breaking down which is advantageous for use with sutures or scaffolds.

To intentionally lower the melt temperature of polycaprolactone, the polymerization reaction conditions can be adjusted to produce a polymer with a lower average molecular weight. Lowering the molecular weight of polycaprolactone directly results in a lower melting point, meaning that a polycaprolactone with a lower molecular weight will melt at a lower temperature. Shorter chains have less intermolecular forces, leading to a reduced melting point. When the molecular weight of polycaprolactone is high, the polymer chains become more entangled with each other, requiring more energy and a higher temperature to overcome these entanglements and melt the material. Lower molecular weight polycaprolactone has shorter chains, which can pack together less efficiently in a crystalline structure, leading to a lower melting point. Reducing the molecular weight can also affect other properties of polycaprolactone, such as its viscosity and mechanical strength. A polycaprolactone is considered a “low-melting” PCL where the melt point is below 45° C.

To crosslink the collagen, collagen is extracted from a source like animal tissue, then exposed to a crosslinking agent, which can be a chemical like glutaraldehyde (GA), 1-ethyl-3-(3-dimethylaminopropyl)carbodiimide (EDC) with N-hydroxysuccinimide (NHS), or utilize physical methods like ultraviolet (UV) irradiation or dehydrothermal (DHT) treatment, which induce crosslinks between collagen molecules, enhancing its stability and mechanical strength. Source material (like bovine skin or tendon) is cleaned and treated with acid to dissolve the collagen fibers. The extracted collagen solution is then purified and concentrated. The crosslinking can be achieved via various methods. Chemical cross linking involves activating carboxyl groups on collagen, allowing them to react with amine groups on other collagen molecules to form covalent bonds. For example, Glutaraldehyde (GA) reacts with amine groups on collagen, forming crosslinks between multiple collagen chains. Genipin, a natural crosslinker also reacts with amine groups on collagen. Physical crosslinking with UV irradiation by exposing the collagen solution to UV light can induce crosslinks between collagen molecules. Alternatively, Dehydrothermal treatment (DHT) involves heating the collagen solution under controlled conditions to cause the crosslinking. The preferred method of crosslinking is one that does not involve toxic residues such as UV treatment or crosslinking with a non-toxic crosslinker such as glyoxal.

The low-melt polycaprolactone is then mixed with powdered collagen, and melted together and extruded to form a collagen-polycaprolactone composite core. The polycaprolactone can be processed to lower its molecular weight in order to reduce the melt temperature. The loading ratio of collagen to polycaprolactone is 5% to 80%. The composite is extruded into a filament and over-braided with polyester and high molecular weight polyethylene and/or polyester. The combined freeze dried collagen powder and low melt temp polycaprolactone results in a composition that can be extruded into a filament. It is formed into a 0.2 to 0.5 mm filament that is overbraided with the polyester. The resulting suture with a core becomes the scaffold so that cells can grow into the scaffold. Micronized salt may optionally be added to the mix which is later washed out either in processing or in vivo to form controlled porosity of the composite in the range of 5 to 300 microns.

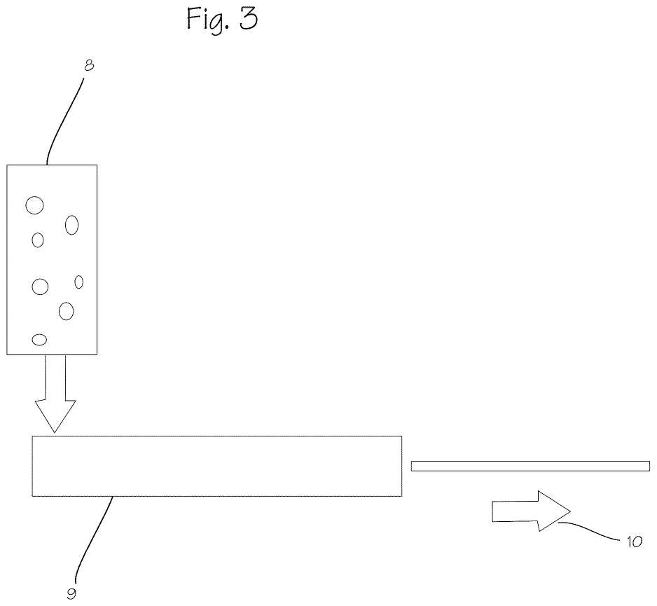

FIG. 3 illustrates the method for extruding the collagen-polycaprolactone filament for additive manufacturing. The material may be extruded in a filament on to a cooling plate or cooled uptake roller operating at less than 45° C. The freeze-drying of the collagen in the matrix produces porosity in the composite material which enables cell attachment and proliferation. The resulting composite filament 9 may be loaded with additives such as hydroxyapatite (HA) to help stimulate bone growth.

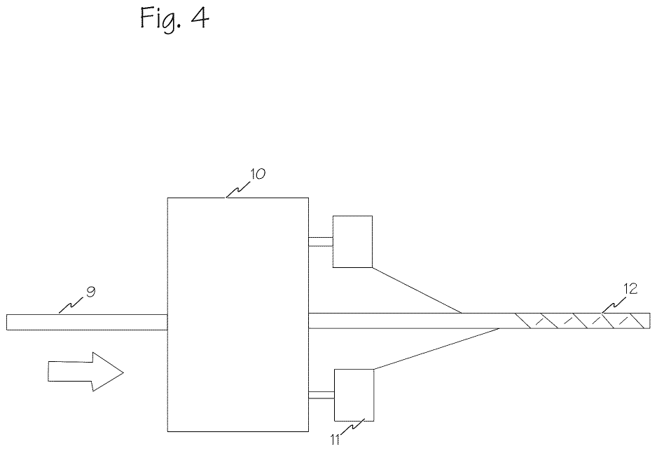

FIG. 4 illustrates the suture overbraid method. The collagen-polycaprolactone composite filament 9 is over-braided with polyester, polyethylene, or a combination of the two to form an absorbable and non-absorbable composite suture with sufficient strength to perform orthopedic tendon repairs. The collagen-polycaprolactone composite filament is loaded into a braiding machine 10 including bobbins 11. The collagen-polycaprolactone composite is loaded into the braiding machine and feed through the center of the braiding machine. The polyester, polyethylene or other material is then wound onto the bobbins evenly and securely, and tensioned on the bobbin to form the braided suture 1. The over braiding is done with standard suture braiding machines. The over braid may also be done with PCL, PDO, or PLLA fibers to make a fully absorbable suture construct.

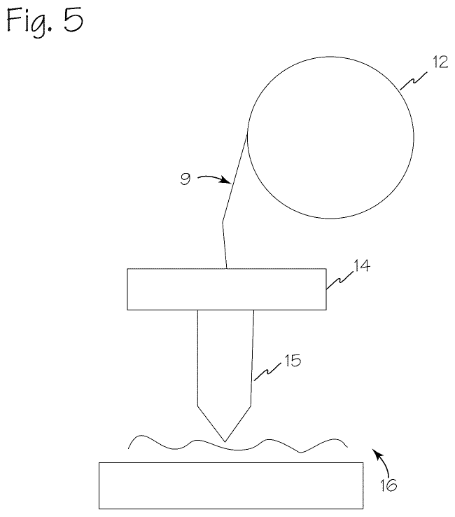

FIG. 5 illustrates a method of 3D printing a composite filament for a surgical suture. The collagen-polycaprolactone composite filament 9 is formed as described above. The filament is then loaded onto printer spool 12 and fed through the printer via a filament feed 13. Printing the begins via the 3D printer X/Y/Z motion control unit 14. The thermal print nozzle 15 must operate at 45° C. or less to produce a 3D printed object made from collagen-polycaprolactone composite if the collagen is uncrosslinked, or below 100° C. when the collagen is crosslinked 16. The polycaprolactone material may be micronized to a diameter of 10-300 microns making it suitable for sintering. The compounded polycaprolactone and collagen material may be melt processed, or may be compression molded or sintered together. The sintering may be performed by heat, heat and compression, or laser sintering in a 3D printing powder-bed printer. The composite may also be processed with melt electrospinning.

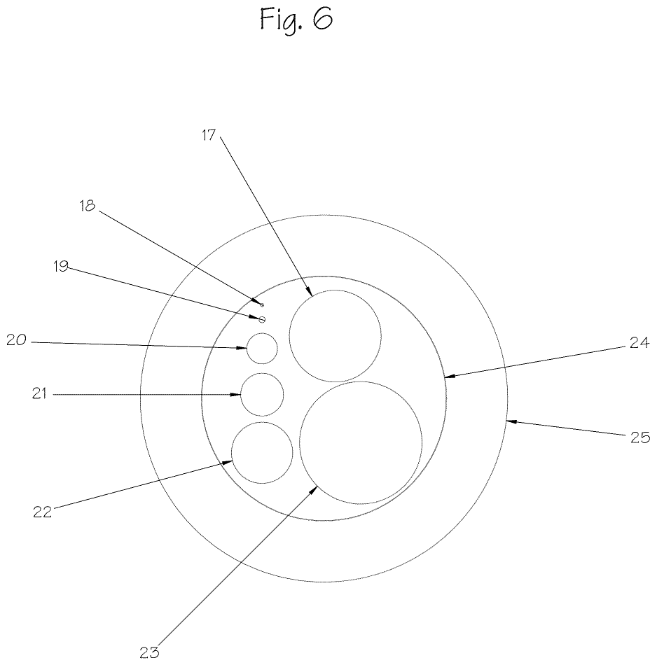

FIG. 6 illustrates the collagen particle size relative to the core size in comparison. For a core diameter of 0.35 to 0.4 mm, the preferred collagen particle size is 50-100 micron in order to maintain enough polymer material in the matrix for structural integrity. In this figure, several collagen particles are illustrated for reference. Item 17 is particle of 150 microns diameter, Item 18 is a particle of 5 microns diameter, Item 19 is a particle of 10 microns diameter, Item 20 is a particle of 50 microns diameter, Item 21 is a particle of 70 microns diameter. Item 22 illustrates a core diameter of 100 microns for the nominal core with Item 23 is 200 microns. The nominal PCL core size 24 is 400 microns in diameter with the overbraid diameter suture size 25 of 600 microns diameter.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

We claim:

1. A surgical suture construct comprising:
a composite filament forming the core of the surgical suture construct, the composite formed by an uncrosslinked collagen blended with polycaprolactone that is extruded into a composite filament at a processing temperature below 45° C.;
a plurality of braided polyester and polyethylene fibers surrounding the composite filament to form the surgical suture;
wherein the composite filament is configured to be a scaffold for tissue ingrowth and the braided fibers provide mechanical reinforcement to the surgical suture construct.

2. A surgical suture construct comprising:
a composite filament forming the core of the surgical suture construct, the composite formed by a crosslinked collagen blended with polycaprolactone that is extruded into a composite filament at a processing temperature below 100° C.;
a plurality of braided polyester and polyethylene fibers surrounding the composite filament to form the surgical suture;
wherein the composite filament is configured to be a scaffold for tissue ingrowth and the braided fibers provide mechanical reinforcement to the surgical suture construct.

3. A bioabsorbable composite material comprising:
uncrosslinked collagen; and
a polymer blended with the uncrosslinked collagen;
wherein the polymer has a melt temperature that is lower than a denaturing temperature of the uncrosslinked collagen.

4. A bioabsorbable composite material comprising:
crosslinked collagen; and
a polymer blended with the crosslinked collagen;
wherein the polymer has a melt temperature that is lower than a denaturing temperature of the crosslinked collagen.
